(19) United States
(12) Patent Application Publication
(10) Pub. No.: US 2026/0209197 A1
(43) Pub. Date: Jul. 23, 2026

(54) CONTINUOUS METHOD FOR PRODUCING LACTONES

(71) Applicant: Massachusetts Institute of Technology, Cambridge, MA (US)

(72) Inventors: Alexander Khechfe, Somerville, MA (US); Lucas Allen, Baston, MA (US); Yurly Roman, Cambridge, MA (US)

(21) Appl. No.: 19/279,265

(22) Filed: Jul. 24, 2025

Related U.S. Application Data
(60) Provisional application No. 63/675,565, filed on Jul. 25, 2024.

(51) Int. Cl. C07D 309/30; B01J 23/02

(52) U.S. Cl. CPC C07D 309/30; B01J 23/02

(57) ABSTRACT

Disclosed are methods of producing an α,β-unsaturated lactone from combining a lactone and a carbonyl compound in a reactor with a metal oxide catalyst.

### RELATED APPLICATIONS

This application claims the benefit of priority to U.S. Provisional Application No. 63/675,565 filed Jul. 25, 2024.

### GOVERNMENT SUPPORT

This invention was made with government support under grant numbers G-64020-01 and DE-SC0022290 awarded by the U.S. Department of Energy. The government has certain rights in this invention.

### BACKGROUND

Plastics have found use in nearly every aspect of modern life due to their light weight, chemical stability, and tunable mechanical and optical properties based on the choice of monomers, crosslinkers, and additives. Due to these advantageous qualities, tens of thousands of tons of plastics are expected to be produced in the coming decades. At the same time, over 6300 Mt of plastic waste have been generated since 1950 with 60% having been discarded. As the use of plastics continues to grow, the environmental impact of their disposal into the environment and fossil fuel emissions during production has grown from concern into crisis. Mechanical recycling technologies have only been able to address ~10% of plastic waste production, necessitating new methods to address the growing environmental catastrophe. Chemical recycling, as a method to selectively break a polymer back down to its respective monomer by the addition of heat and/or a catalyst, has recently attracted attention as a means to deconstruct plastic materials that may not be suitable for mechanical recycling. In particular, poly(alpha-methylene-delta-valerolactone) (PMVL) has been shown to be highly chemically recyclable. Accordingly, sustainable and industrial-scale production of the alpha-methylene-delta-valerolactone monomer is needed.

### SUMMARY OF THE INVENTION

In one aspect the present disclosure provides a method of producing an α,β-unsaturated lactone, comprising:
i. providing a reactor containing a catalyst, wherein the catalyst comprises a metal oxide catalyst; and
ii. contacting the catalyst with a lactone and a carbonyl compound in the reactor, thereby producing the α,β-unsaturated lactone.

### BRIEF DESCRIPTION OF THE DRAWINGS

FIG. 1 shows the general reaction pathway for α-methylene-δ-valerolactone (MVL) synthesis.

FIG. 2 shows the Carbon Dioxide Temperature Programmed Desorption (CO₂ TPD) traces for MgO/SiO₂, CaO/SiO₂ and BaO/SiO₂ catalysts.

FIG. 3 shows (a)¹H NMR spectrum of product mixture from CaO flow reaction in region of interest taken in deuterated acetone solvent with predicted proton chemical shifts. (b) Gas Chromatography-Mass Spectrometry (GC-MS) spectrum of product observed at a retention time of 5.7 min, determined to be MVL.

FIG. 4 shows product distributions and δ-valerolactone (DVL) conversions as functions of contact time for (a) MgO/SiO₂, (b) CaO/SiO₂ and (c) BaO/SiO₂ (613 K, 0.1 g catalyst, 0.4 kPa DVL, 1.2 kPa formaldehyde (FA), 101 kPa, balance N₂).

FIG. 5. shows the product distribution and DVL conversion over CaO/SiO₂ as a function of (a) reaction temperature (contact time of 0.27 h, 0.4 kPa DVL, 1.2 kPa FA, 0.1 g catalyst, 101 kPa) and (b) FA:DVL ratio (613 K, contact time of 0.27 h, 0.4 kPa DVL, 100 mg catalyst, 101 kPa).

FIG. 6 shows MVL production rate (per g_{cat}) as a function of time on stream (613 K, contact time of 0.27 h, 0.4 kPa DVL, 1.2 kPa FA, 101 kPa). Dashed lines represent a regeneration in 50 mL min⁻¹ of air for 4 h.

FIG. 7 shows High-Angle Annular Dark-Field Scanning Transmission Electron Microscopy (HAADF-STEM) images (a) MgO, (b) CaO, and (c) BaO supported on SiO₂.

FIG. 8 shows two representative Transmission Electron Microscopy (TEM) images of the 5 wt % MgO/SiO₂ sample. Smaller dark points are MgO nanoparticles, while larger dark spots are stacked layers of SiO₂ support.

FIG. 9 shows two representative TEM images of the 5 wt % CaO/SiO₂ sample. Smaller dark points are CaO nanoparticles, while larger dark spots are stacked layers of SiO₂ support.

FIG. 10 shows two representative TEM images of the 5 wt % BaO/SiO₂ sample. Smaller dark points are BaO nanoparticles, while larger dark spots are stacked layers of SiO₂ support.

FIG. 11 shows X-ray Diffraction (XRD) patterns of (a) MgO/SiO₂, (b) CaO/SiO₂, and (c) BaO/SiO₂. The only observable feature is a broad peak near 220 corresponding to SiO₂.

FIG. 12 shows Thermogravimetric Analysis (TGA) (45 mL min⁻¹ air, 5 cm³ min⁻¹N₂, 1 K/min ramp) of spent MgO/SiO₂, CaO/SiO₂, and BaO/SiO₂ catalysts after 6-8 h on stream (reaction conditions: 613 K, 0.4 kPa DVL, 1.2 kPa FA, 101 kPa).

FIG. 13 shows a sample gas chromatogram (GC) from a typical gas-phase injection (0.1 g CaO, 613 K, 0.4 kPa DVL, 1.2 kPa FA, contact time of 0.60 h) showing the reaction compounds and their retention times.

FIG. 14 shows the Nuclear Magnetic Resonance (NMR) proton spectrum of product mixture from CaO flow reaction in region of interest taken in deuterated acetone solvent. Labeled MVL molecule shows predicted chemical shifts from ChemDraw, which are consistent with the observed singlets at ~5.5 ppm and 6.3 ppm. One set of triplets near 4.4 ppm corresponds to protons from unreacted DVL.

FIG. 15 shows the GC-MS spectrum of product observed at a retention time of 5.7 min, determined to be α-methylene-δ-valerolactone (MVL).

FIG. 16 shows the GC-MS spectrum of product observed at a retention time of 3.9 min, determined to be γ-valerolactone (GVL).

FIG. 17 shows the GC-MS spectrum of product observed at a retention time of 5.4 min, suspected to be α-methyl-δ-valerolactone (methy-DVL).

FIG. 18 shows the representative GC-MS spectrum of product eluting at >10 min retention times that persist even in the absence of vaporized liquid feeds, indicating that they are significantly less volatile than any other compounds in the reaction system. Due to the similar features at m/z≤100 with DVL alongside the presence of larger peaks, these products are suspected to be dimers that are likely formed from the aldol condensation of DVL or MVL with other compounds.

### DETAILED DESCRIPTION OF THE INVENTION

The high stability of commodity polymers, including polyolefins and polyacrylates, leads to difficulty in developing chemical recycling technologies for these materials. While a great deal of work has been done in breaking down polyethylene and polypropylene using hydrogenolysis, hydrocracking, or pyrolysis, these processes rarely yield the starting monomer in a single step. Several processes have been studied for deconstructing poly(methyl-methacrylate) (PMMA) into methyl-methacrylate (MMA) with more success, but these processes generally require temperatures above 723 K and careful control of PMMA residence time to avoid side reactions that reduce MMA yields. Improving chemical recyclability of these polymers will require redesigning their molecular structures starting from the monomer backbones.

Poly-hydroxyalkanoates (PHA) have received much attention in recent years as chemically recyclable alternatives to many commodity plastics. A subset of these PHAs include alternatives to PMMA using bio-based vinyl-lactones as monomers. A number of polymers based on 5-membered lactone rings have been studied, especially those with methylene substitutions at the α-position. PHAs based on the six-membered lactone ring, δ-valerolactone (DVL), have also been studied. Addition of a vinyl group to the α-position yields α-methylene-δ-valerolactone (alpha-methylene-delta-valerolactone or MVL), which can be polymerized via vinyl-addition polymerization (VAP) or ring-opening polymerization (ROP). (PMVL)_{VAP} has been shown in the past to be highly chemically recyclable, with a 96% yield of MVL after heating at 423 K at 0.01 torr for 6 h using a stannous octoate catalyst. (PMVL)_{R}OP with a molecular weight of 74.6 kDa has also been demonstrated as a mechanically tough, chemically recyclable thermoplastic with similar mechanical properties to commodity polyolefins.

While PMVL shows great promise as a chemically recyclable polymer, synthesis of MVL has only been demonstrated in batch at the lab-scale (~10-100 g). Typically, this synthesis is achieved by the reaction of DVL with ethyl formate and paraformaldehyde using stoichiometric, non-biobased bases, such as NaH, resulting in both high costs and hazardous waste streams. Sustainable and industrial-scale production of PMVL will almost certainly require the development of a continuous, catalytic process for the synthesis of the MVL monomer. Continuous synthesis of the MVL isomer, α-methylene-γ-valerolactone (MGVL), has been reported in the vapor-phase via the aldol condensation of γ-valerolactone (GVL) and formaldehyde (FA) over silica supported alkali and alkaline earth oxide catalysts, with BaO/SiO₂ showing >95% selectivity to MGVL at 613 K. The same reaction was performed in the liquid-phase over supported Cs oxides, with the highest MGVL production rate observed on a 5 wt % Cs oxide supported on a hierarchical beta zeolite (553-583 K, methyl-tetrahydrofuran solvent). This chemistry has been further explored in the patent literature, for instance, in U.S. Pat. Nos. 7,199,254 B2 and 7,164,032 B2, the entire teachings of both of which are incorporated herein by reference in their entirety. Despite these developments, continuous processes for the industrial-scale synthesis of MVL monomer are still needed.

### Definitions

Unless defined otherwise, all technical and scientific terms used herein have the meaning commonly understood by a person skilled in the art of the present disclosure. The following references provide one of skill with a general definition of many of the terms used in this disclosure: Singleton et al., Dictionary of Microbiology and Molecular Biology (2nd ed. 1994); The Cambridge Dictionary of Science and Technology (Walker ed., 1988); The Glossary of Genetics, 5th Ed., R. Rieger et al. (eds.), Springer Verlag (1991); and Hale & Marham, The Harper Collins Dictionary of Biology (1991). As used herein, the following terms have the meanings ascribed to them below, unless specified otherwise.

The transitional term “comprising”, which is synonymous with “including,” “containing,” or “characterized by,” is inclusive or open-ended, and does not exclude additional, unrecited elements or method steps. The transitional phrase “consisting of” excludes any element, step, or ingredient not specified in the claim. The transitional phrase “consisting essentially of” limits the scope of a claim to the specified materials or steps and those that do not materially affect the basic and novel characteristic(s) of the claimed invention.

Ranges provided herein are understood to be shorthand for all of the values within the range. For example, a range of 1 to 50 is understood to include any number, combination of numbers, or sub-range from the group consisting 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50.

Unless specifically stated or obvious from context, as used herein, the term “or” is understood to be inclusive; any species linked by “or” also includes any mixture thereof. Unless specifically stated or obvious from context, as used herein, the terms “a”, “an”, and “the” are understood to be singular or plural.

Unless specifically stated or obvious from context, as used herein, the term “about” is understood as within a range of normal tolerance in the art, for example within 2 standard deviations of the mean. “About” can be understood as within 10%, 9%, 8%, 7%, 6%, 5%, 4%, 3%, 2%, 1%, 0.5%, 0.1%, 0.05%, or 0.01% of the stated value. Unless otherwise clear from context, all numerical values provided herein are modified by the term about.

As used herein, the term “alkali earth metal” refers to elements found in group 2 of the periodic table of the elements, e.g., Mg, Ca, and Ba.

As used herein, the term “oxide” refers to a compound comprising an oxygen element and another chemical element, e.g., SiO₂, MgO, CaO, BaO, and Cs₂O.

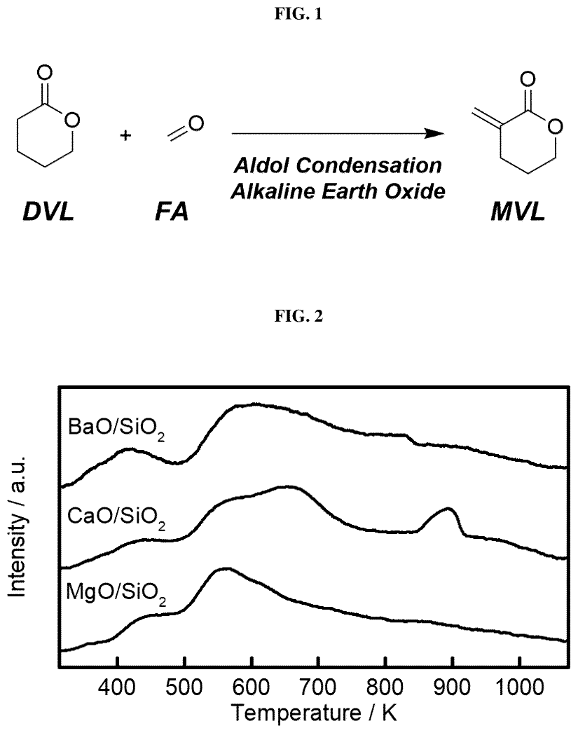

In some embodiments, disclosed herein is a method of aldol condensation over supported alkaline earth oxides for the production of MVL (FIG. 1).

One aspect of the disclosure herein is a method of producing an α,β-unsaturated lactone, comprising:
i. providing a reactor containing a catalyst, wherein the catalyst comprises a metal oxide catalyst; and
ii. contacting the catalyst with a lactone and a carbonyl compound in the reactor, thereby producing the α,β-unsaturated lactone.

In some embodiments of the disclosed method, the lactone is δ-valerolactone (DVL).

In some embodiments of the disclosed method, the carbonyl compound is formaldehyde or acetaldehyde.

In some embodiments of the disclosed method, the α,β-unsaturated lactone is α-methylene-δ-valerolactone (MVL).

In some embodiments of the disclosed method, the metal oxide catalyst comprises an alkali metal or alkali earth metal oxide, or a combination of them.

In some embodiments of the disclosed method, the metal oxide catalyst comprises lithium, sodium, potassium, beryllium, strontium, magnesium, calcium, barium, cesium, or a combination of any of them. In some embodiments of the disclosed method, the metal oxide catalyst comprises magnesium, calcium, barium, or cesium, or a combination of any of them. In some embodiments of the disclosed method, the metal oxide catalyst comprises MgO, BaO, CaO, or a combination of any of them. In a preferred embodiment of the disclosed method, the metal oxide catalyst comprises CaO. In some embodiments of the disclosed method, the metal oxide catalyst loading on the oxide support is 0.5-20 wt %. In certain embodiments, the metal oxide catalyst loading on the oxide support is 3-10 wt %. In certain embodiments, the metal oxide catalyst loading on the oxide support is about 1 wt %, about 2 wt %, about 2.5 wt %, about 2.75 wt %, about 3 wt %, about 3.25 wt %, about 3.5 wt %, about 3.75 wt %, about 4 wt %, about 4.25 wt %, about 4.5 wt %, about 4.75 wt %, about 5 wt %, about 5.25 wt %, about 5.5 wt %, about 5.75 wt %, about 6 wt %, about 7 wt %, about 8 wt %, about 9 wt %, or about 10 wt %. In certain preferred embodiments, the metal oxide catalyst loading on the oxide support is about 2-8 wt %.

In some embodiments of the disclosed method the catalyst further comprises an oxide support.

In some embodiments, the metal oxide catalyst is deposited on the oxide support.

In some embodiments of the disclosed method, the oxide support is silica, alumina, titanium oxide, zirconium oxide, carbon, or a combination of those. In certain embodiments of the disclosed method, the oxide support is SiO₂.

In certain embodiments of the disclosed method, the catalyst comprises magnesium oxide deposited on a SiO₂ support. In certain embodiments of the disclosed method, the catalyst comprises calcium oxide deposited on a SiO₂ support. In certain embodiments of the disclosed method, the catalyst comprises barium oxide deposited on a SiO₂ support.

In certain embodiments of the disclosed method, the catalyst comprises CaO on a SiO₂ support at a loading of 2-8 wt %. In certain embodiments of the disclosed method, the catalyst comprises BaO on a SiO₂ support at a loading of 2-8 wt %. In certain embodiments of the disclosed method, the catalyst comprises MgO on a SiO₂ support at a loading of 2-8 wt %.

In certain embodiments of the disclosed method, the molar ratio of lactone to carbonyl compound is about 1:1 to about 10:1. In certain embodiments of the disclosed method, the molar ratio of lactone to carbonyl compound is about 3:1 to about 6:1.

In certain embodiments of the disclosed method, the molar ratio of lactone to carbonyl compound is 6:1, 5:1, 4:1, 3.5:1, 3:1, 2.5:1, 2:1, 1.75:1, 1.5:1, 1.4:1, 1.3:1, 1.2:1, or 1.1:1.

In some embodiments of the disclosed method, the reactor is heated to about 200-500° C. In some embodiments of the disclosed method, the reactor is heated to about 300-400° C.

In certain embodiments of the disclosed method, the reactor is heated to about 300° C., about 320° C., about 340° C., about 350° C., about 360° C., about 380° C., or about 400° C.

In some embodiments of the disclosed method, the reactor is a batch reactor.

In some embodiments of the disclosed method, the reactor is a flow reactor.

In certain embodiments of the disclosed method, the reactor is a fixed bed reactor. In certain embodiments of the disclosed method, the reactor is a fixed bed reactor; and the catalyst is mixed with silicon carbide as a diluent.

In some embodiments of the disclosed method, the method further comprises calcining the catalyst at about 350-500° C., thereby regenerating the catalyst. In some embodiments of the disclosed method, the method further comprises calcining in air. In some embodiments of the disclosed method, the method further comprises calcining for between one and five hours. In some embodiments of the disclosed method, the method further comprises calcining in air for about four hours.

In certain preferred embodiments of the disclosed method, the lactone and a carbonyl compound are introduced in the reactor together as a pre-heated mixed gas stream.

In some embodiments of the disclosed method, the α,β-unsaturated lactone is produced with conversion of lactone of greater than about 50%. In certain embodiments of the disclosed method, the conversion of lactone to α,β-unsaturated lactone is about 20%, about 25%, about 30%, about 35%, about 40%, about 45%, about 50%, about 55%, or about 60%.

In some embodiments of the disclosed method, the selectivity of the method toward formation of the α,β-unsaturated lactone relative to other reaction products is greater than 50%.

In some embodiments of the disclosed method, the other reaction products comprise α-methylene-γ-valerolactone (MGVL), γ-valerolactone (GVL), and/or α-methyl-δ-valerolactone (methyl-DVL).

In some embodiments of the disclosed method, the α,β-unsaturated lactone is produced with selectivity of about 80% to about 100% relative to other reaction products. In certain embodiments of the disclosed method, the α,β-unsaturated lactone is produced with selectivity of about 80%, about 85%, about 90%, about 92%, about 93%, about 94%, about 95%, about 96%, about 97%, about 98%, or about 99%. In certain embodiments of the disclosed method, the α,β-unsaturated lactone is produced with selectivity of about 95% relative to other reaction products.

In some embodiments, the present disclosure relates to the aldol condensation of DVL with FA over a supported metal oxide for the production of MVL (Scheme 1).

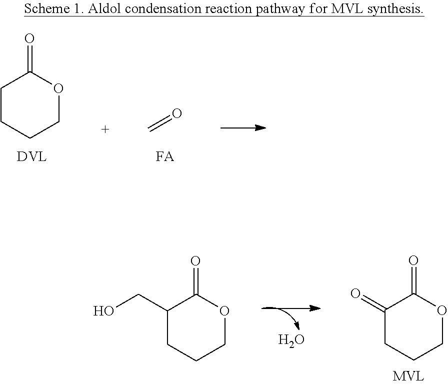

One aspect of the disclosure is a method of producing α-methylene-δ-valerolactone, comprising:
a) providing a heated reactor containing a metal oxide catalyst deposited on an oxide support; and
b) introducing into the reactor formaldehyde and δ-valerolactone.

In one embodiment of the disclosed method, the metal oxide catalyst comprises an alkali metal or alkali earth metal oxide, or a combination of them.

In one embodiment of the disclosed method, the metal oxide catalyst comprises magnesium, calcium, barium, or cesium, or a combination of any of them.

In one embodiment of the disclosed method, the metal oxide catalyst loading on the oxide support is 0.5-20 wt %.

In one embodiment of the disclosed method, the oxide support is silica, alumina, or a combination of any of them.

In one embodiment of the disclosed method, the molar ratio of formaldehyde to δ-valerolactone is at least 1:1.

In one embodiment of the disclosed method, the formaldehyde is in molar excess relative to the δ-valerolactone.

In one embodiment of the disclosed method, the reactor is heated to about 200-500° C.

In one embodiment of the disclosed method, the reactor is heated to about 300-400° C.

In one embodiment of the disclosed method, the reactor is a batch reactor.

In one embodiment of the disclosed method, the reactor is a flow reactor.

In one embodiment of the disclosed method, further comprising regenerating the metal oxide catalyst via calcination at about 350-500° C. for at least four hours.

In one embodiment of the disclosed method, the formaldehyde and δ-valerolactone are introduced together.

In one embodiment of the disclosed method, the formaldehyde and δ-valerolactone are introduced separately.

One aspect of the disclosure herein is a method of catalyzing an aldol condensation reaction, comprising:
a) providing a reactor containing a metal oxide catalyst deposited on an oxide support; and
b) introducing into the reactor a cyclic ester (lactone) and a carbonyl compound.

In one embodiment of the disclosed method, the cyclic ester is δ-valerolactone.

In one embodiment of the disclosed method, the carbonyl compound is formaldehyde or acetaldehyde.

In one embodiment of the disclosed method, the metal oxide catalyst comprises an alkali metal or alkali earth metal oxide, or a combination of them.

In one embodiment of the disclosed method, the metal oxide catalyst comprises magnesium, calcium, barium, or cesium, or a combination of any of them.

In one embodiment of the disclosed method, the metal oxide catalyst loading on the oxide support is 0.5-20 wt %.

In one embodiment of the disclosed method, the oxide support is silica, alumina, or a combination of any of them.

In one embodiment of the disclosed method, the molar ratio of carbonyl compound to cyclic ester is at least 1:1.

In one embodiment of the disclosed method, the carbonyl compound is in molar excess relative to the cyclic ester.

In one embodiment of the disclosed method, the reactor is heated to about 200-500° C.

In one embodiment of the disclosed method, the reactor is heated to about 300-400° C.

In one embodiment of the disclosed method, the reactor is a batch reactor.

In one embodiment of the disclosed method, the reactor is a flow reactor.

In one embodiment of the disclosed method, the method further comprises regenerating the metal oxide catalyst via calcination at about 350-500° C. for at least four hours.

In one embodiment of the disclosed method, the formaldehyde and δ-valerolactone are introduced together.

In one embodiment of the disclosed method, the formaldehyde and δ-valerolactone are introduced separately.

### Results and Discussion

The presence of alkaline earth metals on the silica supports was confirmed via ICP-MS with their respective weight loadings shown in [symbol] Reported weight loadings correspond to metal loadings on silica. CO₂ is able to bind to basic lattice oxygens on metal oxides and can thus be used to probe the presence and relative strength of basic sites on these materials. CO₂ TPD curves are shown in FIG. 2 and show multiple features, indicating that there are a variety of basic sites available for catalysis on each sample. All three catalysts have weaker sites present with desorption temperatures at 400-450 K. On MgO, this is followed by one additional desorption peak at approximately 570 K. CaO shows a pair of overlapping peaks are centered near 570 K and 650 K, with an additional peak at 890 K. BaO shows a similar broad or overlapping set of peaks between 550 K and 700 K, similar to CaO. No further peaks were detected up to 1073 K. In summary, each catalyst contained a variety of basic sites, with CaO and BaO containing stronger sites than MgO.

TABLE 1
Metal weight loading and average particle
size for each catalyst used in this study.

| | Metal weight | Metal oxide |
|---|---|---|
| Catalyst | loading/% | loading/% |
| MgO/SiO₂ | 3.6 | 6.0% |
| CaO/SiO₂ | 5.2 | 5.8% |
| BaO/SiO₂ | 3.9 | 5.5% |

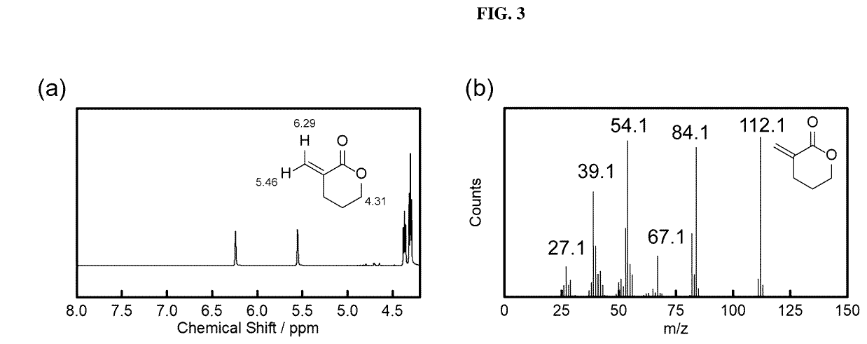

Each of the three catalysts in [symbol] was tested for its activity and selectivity toward MVL production from DVL and FA. Introduction of 0.4 kPa DVL and 1.2 kPa FA at 613 K into a bed of each catalyst resulted in the formation of MVL and small amounts of other side products, including GVL and α-methyl-δ-valerolactone (methyl-DVL). The formation of the desired MVL isomer was confirmed using HNMR by the presence of vinyl proton peaks at 5.5 ppm and 6.3 ppm and by GC-MS (FIG. 3). Small GC chromatogram peaks were observed at higher retention times/oven temperatures that did not respond to changes in reactor conditions, indicating slow deposition of organic species on the catalyst, consistent with similar studies on the aldol condensation of GVL and FA. Conversions decreased sharply on all catalysts for approximately the first 2 hours on stream after feed introduction, after which a second regime of slower deactivation began to occur. In this second regime, conversion generally decreased by ~10% over the span of 6-8 hours under most conditions. Full reactivity was regenerated after calcination in 50 mL min-1 air at 773 K for 4 h. Conversion, selectivity, and rate data reported herein were collected in this second regime of slower, steady deactivation and are not deactivation corrected, with periodic regenerations to restore reactivity. Under conditions of faster deactivation, typically at temperatures above 613 K, conversion and selectivity data is reported from the first hour of the condition before appreciable deactivation occurred.

TABLE 2
Product distributions and DVL conversions, presented in tabulated form, as functions
of contact time for the three catalysts studied in this work. Conditions: 613 K, 0.1
g catalyst, 0.4 kPa DVL, 1.2 kPa FA, 101 kPa, balance N₂.

| | | | | | | Solids/Lost |
|---|---|---|---|---|---|---|
| | | DVL | MVL | GVL | Methyl-DVL | Carbon |
| | Contact | Conversion/ | Selectivity/ | Selectivity/ | Selectivity/ | Selectivity/ |
| Catalyst | Time | % | % | % | % | % |
| MgO/SiO₂ | 0.15 | 29.81 | 75.14 | 24.85 | 0 | 0.01 |
| | 0.30 | 45.53 | 50.18 | 17.92 | 0 | 31.91 |
| | 0.42 | 61.38 | 40.08 | 15.52 | 0 | 44.40 |
| | 0.64 | 73.61 | 27.56 | 12.11 | 0 | 60.33 |
| CaO/SiO₂ | 0.14 | 19.10 | 100 | 0 | 0 | 0 |
| | 0.27 | 40.32 | 96.56 | 0 | 0 | 3.44 |
| | 0.60 | 58.74 | 86.55 | 2.44 | 0.35 | 10.65 |
| | 0.79 | 71.07 | 80.45 | 2.52 | 2.30 | 14.73 |
| BaO/SiO₂ | 0.06 | 24.18 | 100 | 0 | 0 | 0 |
| | 0.16 | 42.71 | 95.28 | 0 | 0 | 4.72 |
| | 0.29 | 61.49 | 83.11 | 0 | 0.60 | 16.29 |
| | 0.78 | 68.13 | 76.70 | 0 | 1.89 | 21.41 |

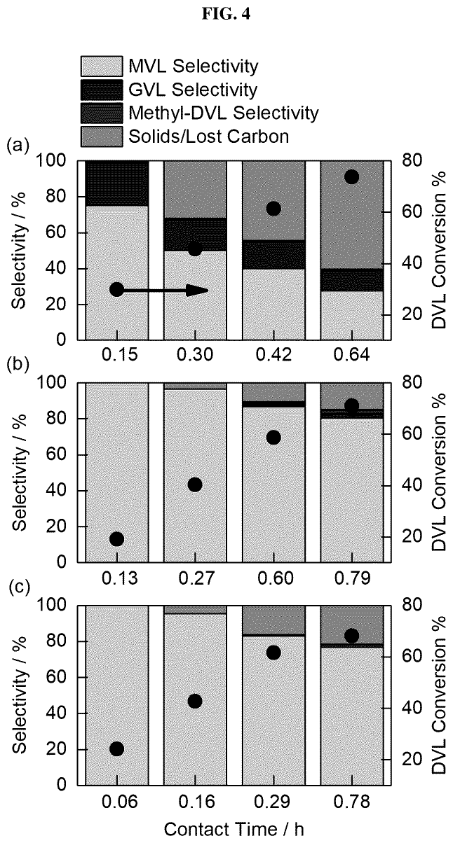

Table 2 and FIG. 4 compare the selectivity of each of the three catalysts to MVL at a range of contact times (defined as the moles of active metal divided by the molar flow rate of DVL) and their corresponding conversions. BaO generally required lower contact times to obtain a given conversion, as compared to MgO or CaO, indicating that the active sites on BaO are more active for aldol condensation. MVL selectivity was higher for both CaO and BaO than over MgO, with CaO featuring 90% selectivity to MVL at 60% conversion. MVL selectivity was only ~40% for MgO at a similar conversion. For MgO, the dominant side product that could be detected in the vapor effluent was GVL (selectivities of 10-25%), indicating the tendency of MgO to catalyze DVL ring-opening reactions in parallel to aldol condensations. DVL is known to isomerize via ring-opening pathways to GVL over Brønsted acidic SiO₂/Al₂O₃. Both aqueous acids and bases are also known to ring-open GVL to form 4-hydroxyvalerates; however, linear analogs of DVL and GVL were not detected in the reactor effluent over our system. While ring-opened linear species may exist on the catalyst surface, they likely only exist as intermediates between DVL and GVL. This ring-opening isomerization activity is attributed to the presence of hydroxyl groups that can be formed in the presence of water, which are believed to be active sites for the isomerization of glucose to fructose in water over MgO. For BaO and CaO, only small amounts (~1-2%) of GVL and methyl-DVL were detected, indicating that carbon loss is likely due to the consumption of DVL and/or MVL to form non-volatiles that cannot be accurately quantified. Methyl-DVL is hypothesized to form via hydrogenation of MVL by trace methanol present in the formalin feed. CaO maintained slightly higher selectivity to MVL at higher DVL conversion than BaO. While this work seeks to elucidate general reactivity trends for this reaction across these materials, the detailed nature of the active sites and mechanisms for the different observed reactions will be the subject of future studies.

CaO was observed to have the highest selectivity at high DVL conversion for this reaction. At conversions <40% for CaO/SiO₂, selectivity to MVL is >95% with no observed side products in the GC. As with the other two materials, increased contact time decreases selectivity to MVL; however, most of the remaining DVL forms non-volatile products that are not detected in the GC. This provides further evidence that MVL is consumed at higher contact times via series reactions with DVL to form non-volatiles. Based on these data, the process can potentially be operated at conversions <40%, and with nearly quantitative yields of MVL.

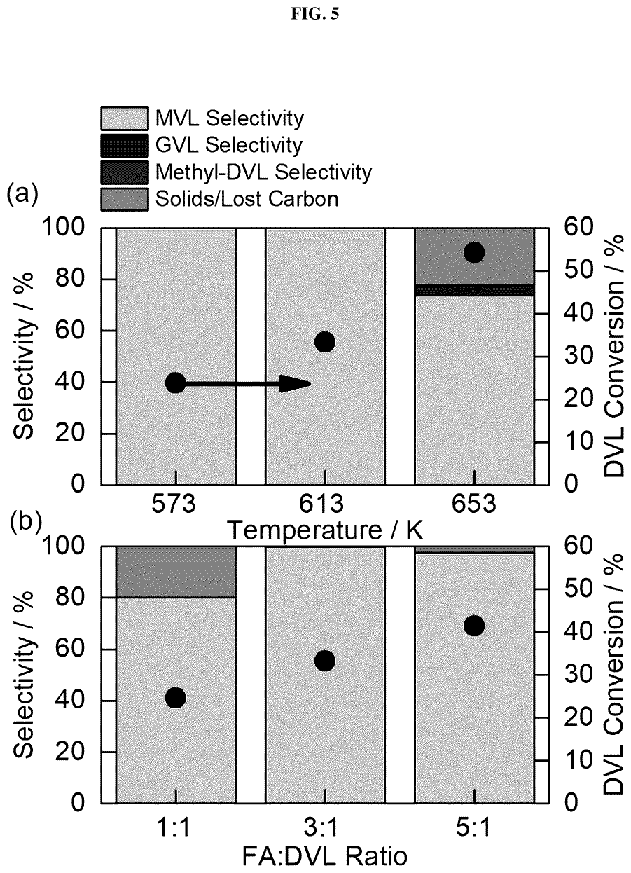

Due to its high selectivity toward MVL at elevated conversions, other process parameters were varied over CaO/SiO₂. The effect of temperature on DVL conversion and MVL selectivity is shown in tabulated form in Table 3 and in FIG. 5 panel A. At a given contact time, DVL conversion increases monotonically with reaction temperature at the expense of MVL selectivity. The effect of temperature on MVL consumption is slightly more pronounced than contact time alone: at 653 K, a DVL conversion of 54% leads to an MVL selectivity of 74%, while a 59% DVL conversion at 613 K yields 86% selectivity to MVL. Side reactions thus begin to play a slightly more significant role in determining product distributions at higher temperatures. Increasing the ratio of FA to DVL to 5:1 increases the conversion of DVL for a given contact time by ~10%, based on Table 4 and FIG. 5 panel B, while maintaining >95% selectivity to MVL. Decreasing the formaldehyde in the feed to a 1:1 ratio with DVL decreases the DVL conversion by ~10% with a concomitant loss in MVL selectivity. In summary, MVL selectivities can be optimized over CaO/SiO₂ at low to moderate DVL conversions with an excess of FA in the feed.

TABLE 3
Product distribution and DVL conversion over
CaO/SiO₂ as a function of reaction temperature, presented
in tabulated form. Conditions: contact time of 0.27
h, 0.4 kPa DVL, 1.2 kPa FA, 0.1 g catalyst, 101 kPa.

| | | | | | Solids/ |
|---|---|---|---|---|---|
| | | | | | Lost |
| | | MVL | GVL | Methyl-DVL | Carbon |
| | DVL | Selec- | Selec- | Selec- | Selec- |
| Temperature | Conversion/% | tivity/% | tivity/% | tivity/% | tivity/% |
| 573 | 23.72 | 100 | 0 | 0 | 0 |
| 613 | 40.32 | 96.56 | 0 | 0 | 3.44 |
| 653 | 54.25 | 73.72 | 3.90 | 0 | 22.38 |

TABLE 4
Product distribution and DVL conversion over CaO/SiO₂ as a function
of FA:DVL ratio, presented in tabulated form. Conditions: 613 K,
contact time of 0.27 h, 0.4 kPa DVL, 100 mg catalyst, 101 kPa.

| | | | | | Solids/ |
|---|---|---|---|---|---|
| | | | | | Lost |
| FA:DVL | | MVL | GVL | Methyl-DVL | Carbon |
| Molar | DVL | Selec- | Selec- | Selec- | Selec- |
| Ratio | Conversion/% | tivity/% | tivity/% | tivity/% | tivity/% |
| 1:1 | 24.55 | 80.14 | 0 | 0 | 19.86 |
| 3:1 | 40.32 | 96.56 | 0 | 0 | 3.44 |
| 5:1 | 41.34 | 97.45 | 0 | 0 | 2.55 |

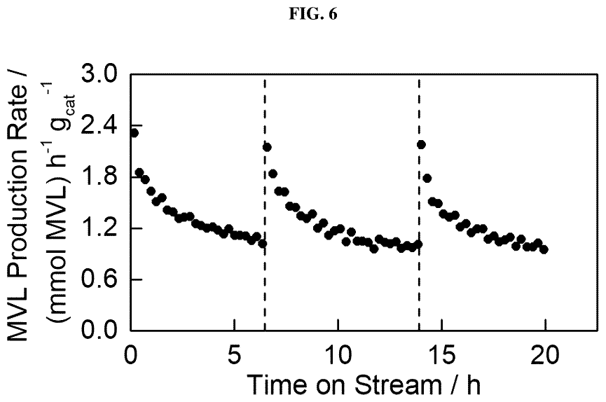

Transient curves showing MVL production rate vs. time on stream over CaO/SiO₂ are shown in FIG. 6, with dashed lines representing a regeneration in 50 mL min-1 air at 773 K for 4 h. After an initial, faster deactivation period during the first ~2 h on stream, the second regime of slow deactivation begins and is maintained for several hours. Regeneration in air restores reactivity, showing that these catalysts are robust to multiple calcination cycles. This also indicates that minimal chemical or structural changes, such as the formation of inactive chemical surfaces or particle sintering, occur during the course of the reaction.

In some embodiments, the present disclosure demonstrates that the acrylic monomer MVL can be continuously produced via the gas-phase aldol condensation of DVL and FA over silica supported alkaline earth oxides. CaO and BaO demonstrated selectivity to MVL>90% at DVL conversions <50%. Selectivity to MVL over CaO generally decreased slightly with increasing conversion and temperature and decreasing FA partial pressures. Selectivity loss was attributed to the presence of series reactions that consume MVL and generate non-volatile lactone dimers at higher conversions (Scheme 2). These non-volatiles lead to slow and steady catalyst deactivation over several hours of operation, although the catalyst activity can be fully regenerated by calcination in air. Future work will entail the engineering of catalysts to minimize the formation of lactone dimers, thereby mitigating selectivity loss and catalyst deactivation.

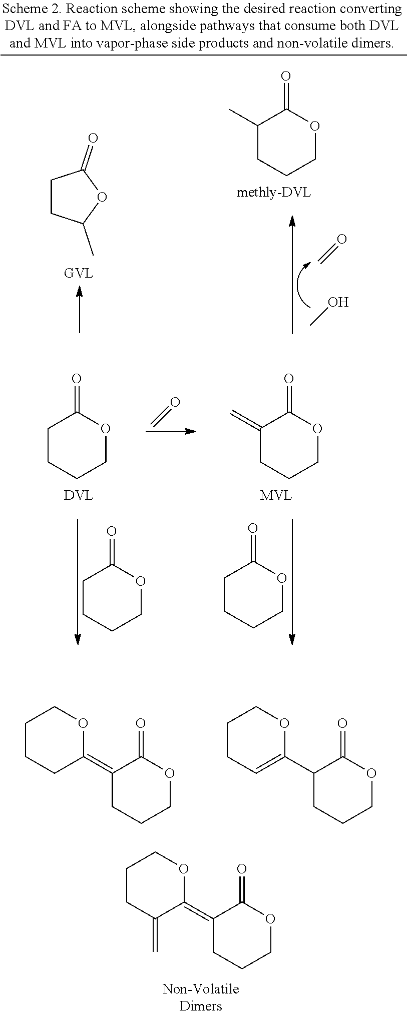

### Advantages and Improvements Over Existing Methods

Existing methods for MVL production rely on the stoichiometric use of bases, such as NaH, and compounds such as ethyl formate that can produce large toxic waste streams. When run under ~30-40% conversion, the only large byproduct of this reaction is water with only small amounts of other organic species.

The continuous, gas-phase synthesis reported here is amenable to scale-up over existing batch processes. The catalysts are expected to be cheap to produce and can be regenerated by simple air treatments, allowing for long lifetimes of the catalyst bed.

The only necessary reactants are DVL, which can be sourced from biomass, and formaldehyde, which is a readily available and cheap chemical feedstock.

### Commercial Applications

The scalable production of MVL opens new avenues for the large-scale production of the chemically recyclable polymers (PMVL)_{VAP} and (PMVL)_{R}OP, which may be of interest to plastics producers seeking to reduce their environmental footprints.

### EXAMPLES

### Catalyst Synthesis

Supported alkaline earth oxide catalysts were prepared via incipient wetness impregnation. Fumed silica (SiO₂, Sigma Aldrich) was used as a support and calcined at 873 K for 6 h (4 K min⁻¹ ramp rate) prior to impregnation. Magnesium acetate tetrahydrate, calcium acetate monohydrate, or barium acetate (all Sigma Aldrich, >99%) were dissolved in enough DI water to saturate the pore volume of a given mass of SiO₂ (~2 g_{water} gSiO₂⁻¹). The metal salt solution was then slowly deposited onto the SiO₂, followed by grinding and mixing in a mortar and pestle. The pre-catalyst was then dried for at least 6 hr in an oven at 373 K, ground again, and calcined at 773 K (ramp rate 4 K min⁻¹) for 6 hr.

### Catalyst Characterization

Weight loadings of Mg, Ca, and Ba on SiO₂ were determined by inductively coupled plasma mass spectrometry (ICP-MS) using an Agilent 7900 ICP-MS instrument. Approximately 10 mg of catalyst powder was added to a 15 mL polypropylene centrifuge tube and dissolved in ~1 mL of 68.0% HNO₃ (Veritas Double Distilled, GFS Chemicals Inc.) overnight, followed by dilution in 2 wt % HNO₃ and filtration through a 0.2 m PTFE syringe filter (VWR) to remove the undissolved SiO₂ support. Solutions were further diluted to obtain metal concentrations of 100-300 ppb for Mg and Ba. Ca solutions were diluted to a lesser extent (30-60 ppm) to monitor ⁴⁴Ca (~2% abundance) due to interference between ⁴⁰Ca and the Ar plasma. Metal concentrations were determined from calibration curves prepared from standard solutions of 1000 ppm of Mg, Ca, and Ba (all TraceCERT, Sigma-Aldrich), diluted in 2 wt % HNO₃.

### Imaging of Supported Catalysts

Scanning transmission electron microscopy (STEM) imaging was performed on a probe-corrected Thermo Fisher Scientific Themis Z G3, operated at 200 kV. Images were collected with a 19 mrad collection angle and either 70-200 mrad (Z-contrast, HAADF) or 35-200 mrad (diffraction contrast). Transmission electron microscopy (TEM) was performed on an FEI Tecnai F20 electron microscope to determine particle size distributions for each sample. Catalysts were dispersed in ethanol and sonicated until well-dispersed and then dropped onto a copper grid with a carbon film. Measurements were performed using an electron detector at a 200 kV acceleration voltage.

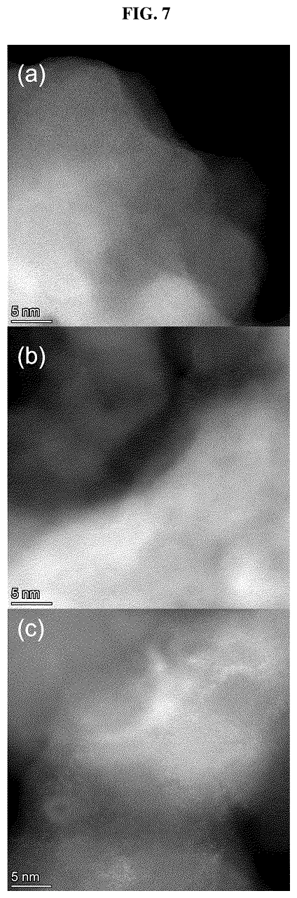

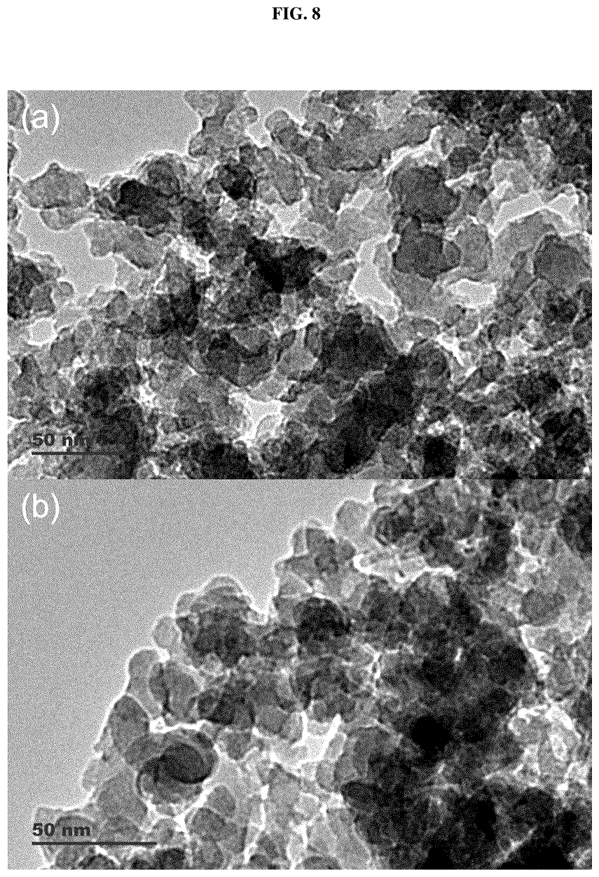

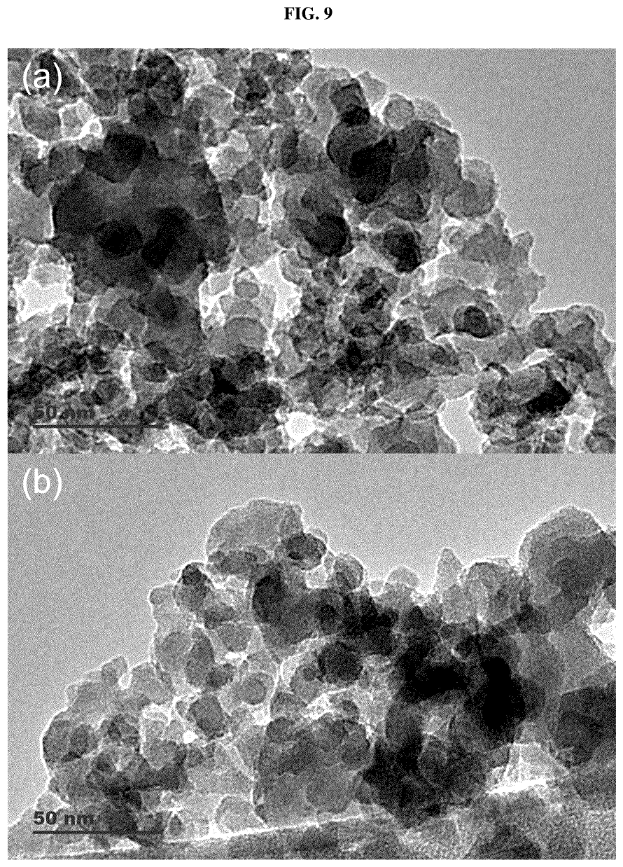

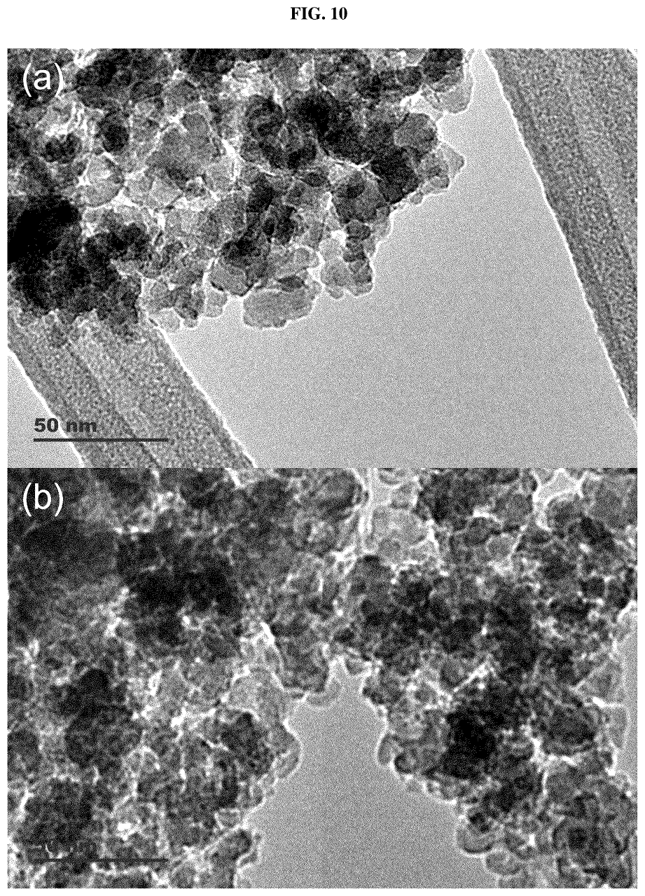

FIG. 7 presents a high-angle annular dark-field scanning transmission electron microscopy (STEM-HAADF) image of the (a) CaO, (b) MgO and (c) BaO over SiO₂ catalyst, captured at a high resolution of 2048×2048 scan points with a magnification of 2.80 million times. On FIG. 7 panel C, barium atoms are distinctly visible as bright white spots, indicating their dispersion across the amorphous silica support without any discernible aggregation into particles. In contrast, for the CaO and MgO catalysts, the metal atoms are not discernible against the SiO₂ background, as shown in FIG. 7 panel A and B. This is attributed to the atomic numbers (Z) of calcium and magnesium being relatively close to that of silicon, which results in insufficient contrast for clear visualization. Among all the collected STEM-HAADF images for the three catalyst samples, only one image, presented in FIG. 7 panel A, reveals the presence of a visible crystalline particle. This particle, identified as a CaO crystal, measures approximately 5 nm in size. Aggregated particles contrasting with SiO₂ were also not observed in TEM (FIGS. 8-10), precluding rigorous particle size distribution determinations.

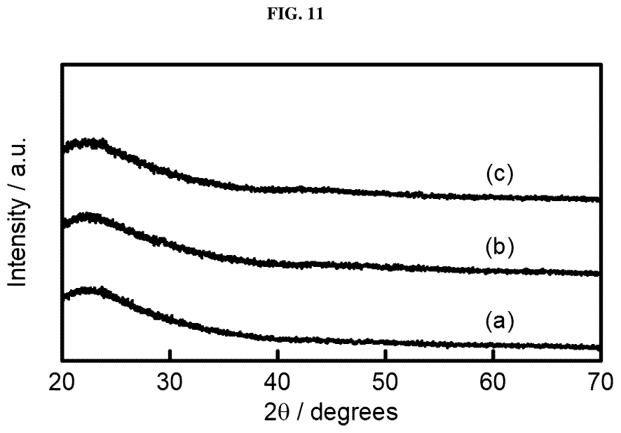

Powder X-ray diffraction (PXRD) was used to determine the crystallinity of metal oxide particles. Patterns were collected on a Bruker D8 diffractometer with a Cu Kα radiation source between 2θ=20-80° and a scan rate of 0.05° s⁻¹. X-ray diffraction patterns for each of the three catalysts only show a broad peak corresponding to SiO₂ at a 20 value of ~22°, corroborating the absence of large crystalline domains over these samples (FIG. 11).

Carbon dioxide temperature programmed desorption (CO₂ TPD) was performed in a Micromeritics Autochem II 2920 unit equipped with a thermal conductivity detector. Approximately 0.15 g of sample was loaded into a quartz U-tube and held in place with plugs of quartz wool on both sides. The sample was pretreated in 50 mL min⁻¹ He at 473 K for 2 h and cooled down to 313 K. The sample was then dosed with 50 mL min⁻¹ of a 1% CO₂ in N₂ and 30 mL min⁻¹ He mixture for 30 minutes, followed by a 10-minute purge in 80 mL min⁻¹ of He. This procedure was repeated an additional two times to saturate the surface with CO₂. The sample was then heated in a 80 mL min⁻¹ He flow while the temperature was ramped from 313 K to 1073 K at a 10 K min⁻¹ ramp rate while monitoring the reactor effluent. See FIG. 2.

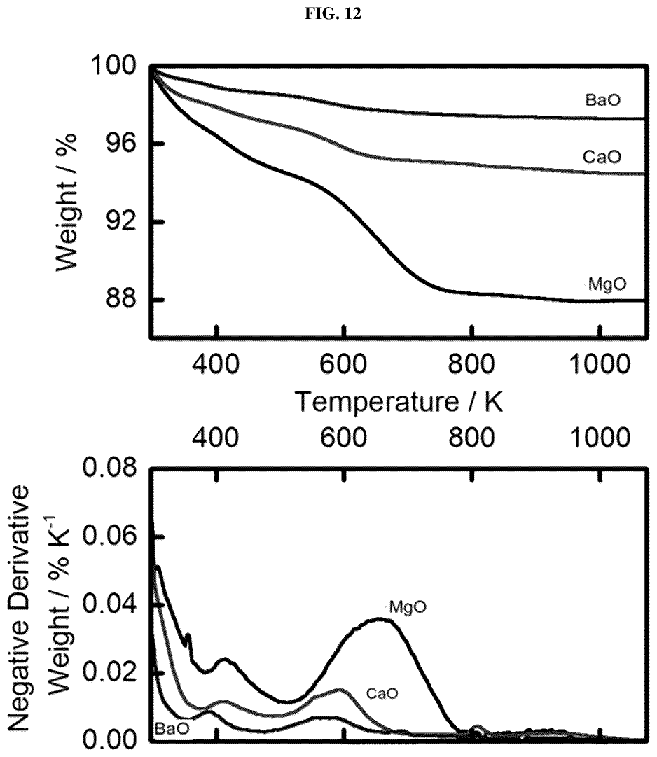

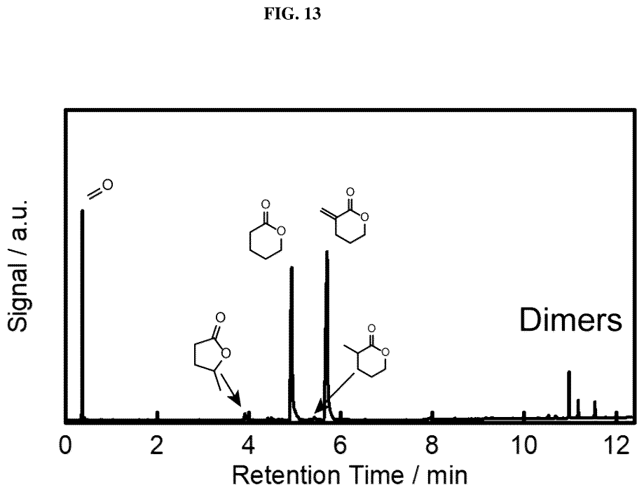

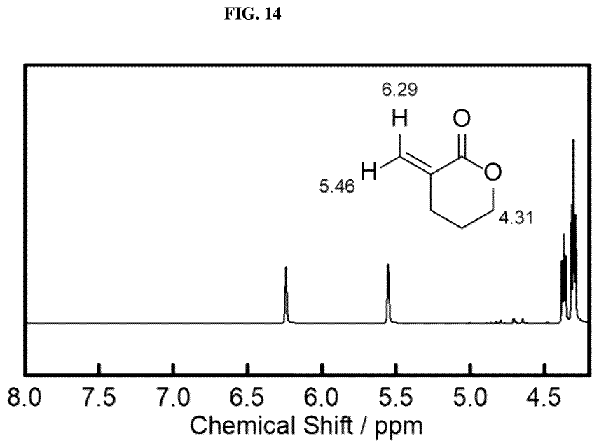

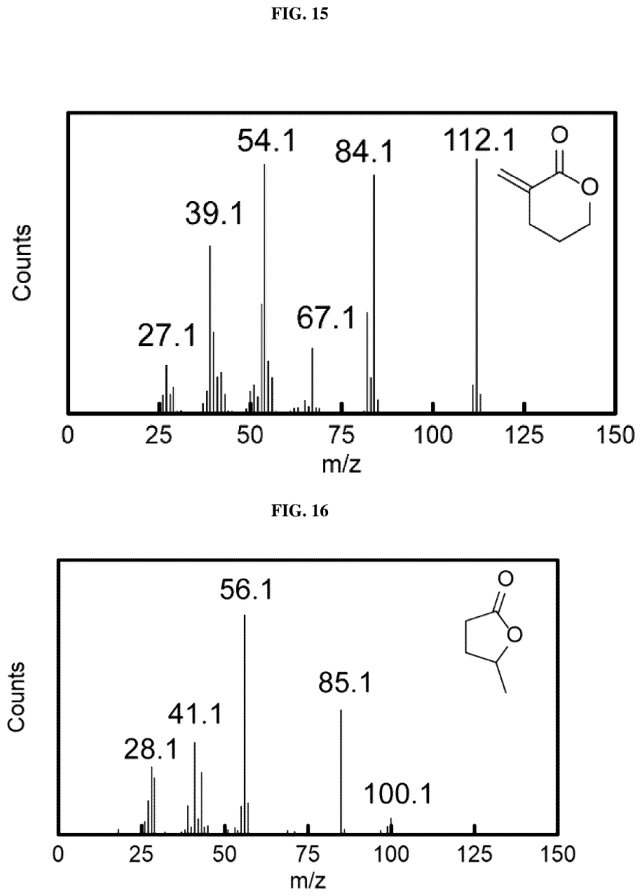

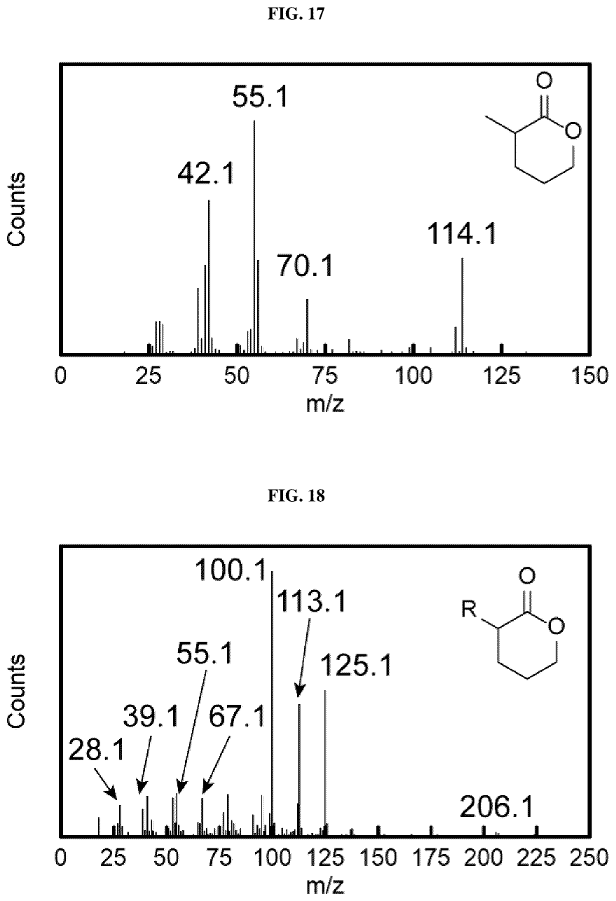

Thermogravimetric analysis (TGA) was performed on a TA Instruments Q500 System. The sample was loaded onto a tared platinum pan and equilibrated at 298 K in 45 mL min⁻¹ of air and 5 mL min⁻¹ N₂. After equilibration, the temperature was ramped at 1 K min⁻¹ to 1073 K. See FIG. 12.

### Continuous Catalytic MVL Synthesis

Aldol condensation between DVL and FA were performed in a gas-phase, fixed bed reactor. Catalysts were pelletized, crushed, and sieved between 40-60 mesh to reduce pressure drop across the catalyst bed. Approximately 0.1 g of catalyst was mixed with 0.4 g of silicon carbide (46 mesh) as a diluent and loaded into a ¼″ 316 stainless steel tube. The catalyst bed was held in place with a plug of quartz wool and topped with ~1.5 g of borosilicate glass beads to facilitate gas-phase mixing. The bed was heated in a furnace (ATS Systems) and temperature was controlled via a K-type thermocouple touching the base of the catalyst bed with Cole-Parmer Digi-Sense Temperature Controller R/S 68900-11.

A mixture of DVL (technical grade, Sigma Aldrich) and formalin (37 wt % FA in water with methanol stabilizer, Sigma Aldrich) were introduced via a Cole-Parmer Masterflex Single-Syringe Infusion Pump with a typical liquid flow rate of ~0.16 mL h⁻¹. The liquid feed was vaporized at ~423 K into a stream of N₂ (Airgas) with a typical flow rate of 50 mL min⁻¹. Reactor effluents were analyzed by an on-line gas chromatograph (Agilent 8890) equipped with a 30 m HP-5MS-UI column and a flame ionization detector (FID). Product selectivities were calculated as

$${S}_{i} = \frac{{\dot{n}}_{ι}}{{\dot{n}}_{DVL , 0} - {\dot{n}}_{DVL}} \times 100 \% ,$$

where Sᵢ is the selectivity of product i, \dot{n}_{DVL,0} is the initial molar flow rate of DVL into the reactor, and \dot{n}_{DVL} and \dot{n}_{ι} are the molar flow rates of DVL and product \dot{ι} in the effluent, respectively. Contact times were calculated as moles of active metal loaded in the reactor divided by the molar flow rate of DVL. Reaction rates were normalized by total mass of supported catalyst unless stated otherwise.

### REFERENCES CITED

(1) Ellen McArthur Foundation. The New Plastics Economy: Rethinking the future of plastics & catalysing action https://emf.thirdlight.com/link/cap0qk3wwwk0-13727v/@/preview/2 (accessed Mar. 22, 2022).
(2) Geyer, R.; Jambeck, J. R.; Law, K. L. Production, Use, and Fate of All Plastics Ever Made. Sci. Adv. 2017, 3 (7). https://doi.org/10.1126/sciadv.1700782.
(3) Cabernard, L.; Pfister, S.; Oberschelp, C.; Hellweg, S. Growing Environmental Footprint of Plastics Driven by Coal Combustion. Nat. Sustain. 2021 52 2021, 5 (2), 139-148. https://doi.org/10.1038/s41893-021-00807-2.
(4) Borrelle, S. B.; Ringma, J.; Lavender Law, K.; Monnahan, C. C.; Lebreton, L.; McGivern, A.; Murphy, E.; Jambeck, J.; Leonard, G. H.; Hilleary, M. A.; et al. Predicted Growth in Plastic Waste Exceeds Efforts to Mitigate Plastic Pollution. Science (80-.). 2020, 369 (6509), 1515-1518. https://doi.org/10.1126/science.aba3656.
(5) Garcia, J. M.; Robertson, M. L. The Future of Plastics Recycling. Science (80-.). 2017, 358 (6365), 870-872. https://doi.org/10.1126/science.aaq0324.
(6) Chu, M.; Liu, Y.; Lou, X.; Zhang, Q.; Chen, J. Rational Design of Chemical Catalysis for Plastic Recycling. ACS Catal. 2022, 12 (8), 4659-4679. https://doi.org/10.1021/acscatal.2c01286.
(7) Tang, X.; Chen, E. Y. X. Toward Infinitely Recyclable Plastics Derived from Renewable Cyclic Esters. Chem 2019, 5 (2), 284-312. https://doi.org/10.1016/j.chempr.2018.10.011.
(8) Zichittella, G.; Ebrahim, A. M.; Zhu, J.; Brenner, A. E.; Drake, G.; Beckham, G. T.; Bare, S. R.; Rorrer, J. E.; Román-Leshkov, Y. Hydrogenolysis of Polyethylene and Polypropylene into Propane over Cobalt-Based Catalysts. JACS Au 2022, 2 (10), 2259-2268. https://doi.org/10.1021/jacsau.2C00402.
(9) Celik, G.; Kennedy, R. M.; Hackler, R. A.; Ferrandon, M.; Tennakoon, A.; Patnaik, S.; Lapointe, A. M.; Ammal, S. C.; Heyden, A.; Perras, F. A.; et al. Upcycling Single-Use Polyethylene into High-Quality Liquid Products. ACS Cent. Sci. 2019, 5 (11), 1795-1803. https://doi.org/10.1021/acscentsci.9b00722.
(10) Chen, L.; Zhu, Y.; Meyer, L. C.; Hale, L. V.; Le, T. T.; Karkamkar, A.; Lercher, J. A.; Gutiérrez, O. Y.; Szanyi, J. Effect of Reaction Conditions on the Hydrogenolysis of Polypropylene and Polyethylene into Gas and Liquid Alkanes. React. Chem. Eng. 2022, 7 (4), 844-854. https://doi.org/10.1039/d1re00431j.
(11) Zhang, F.; Zeng, M.; Yappert, R. D.; Sun, J.; Lee, Y. H.; LaPointe, A. M.; Peters, B.; Abu-Omar, M. M.; Scott, S. L. Polyethylene Upcycling to Long-Chain Alkylaromatics by Tandem Hydrogenolysis/Aromatization. Science (80-.). 2020, 370 (6515). https://doi.org/10.1126/science.abc5441.
(12) Liu, S.; Kots, P. A.; Vance, B. C.; Danielson, A.; Vlachos, D. G. Plastic Waste to Fuels by Hydrocracking at Mild Conditions. Sci. Adv. 2021, 7 (17), 8283-8304. https://doi.org/10.1126/sciadv.abf8283.
(13) Rorrer, J. E.; Ebrahim, A. M.; Questell-Santiago, Y.; Zhu, J.; Troyano-Valls, C.; Asundi, A. S.; Brenner, A. E.; Bare, S. R.; Tassone, C. J.; Beckham, G. T.; et al. Role of Bifunctional Ru/Acid Catalysts in the Selective Hydrocracking of Polyethylene and Polypropylene Waste to Liquid Hydrocarbons. ACS Catal. 2022, 12 (22), 13969-13979. https://doi.org/10.1021/acscatal.2C03596/.
(14) Anuar Sharuddin, S. D.; Abnisa, F.; Wan Daud, W. M. A.; Aroua, M. K. A Review on Pyrolysis of Plastic Wastes. Energy Convers. Manag. 2016, 115, 308-326. https://doi.org/10.1016/j.enconman.2016.02.037.
(15) Kunwar, B.; Cheng, H. N.; Chandrashekaran, S. R.; Sharma, B. K. Plastics to Fuel: A Review. Renew. Sustain. Energy Rev. 2016, 54, 421-428. https://doi.org/10.1016/j.rser.2015.10.015.
(16) Achilias, D. S. Chemical Recycling of Poly(Methyl Methacrylate) by Pyrolysis. Potential Use of the Liquid Fraction as a Raw Material for the Reproduction of the Polymer. Eur. Polym. J. 2007, 43 (6), 2564-2575. https://doi.org/10.1016/j.eurpolymj.2007.02.044.
(17) Godiya, C. B.; Gabrielli, S.; Materazzi, S.; Pianesi, M. S.; Stefanini, N.; Marcantoni, E. Depolymerization of Waste Poly(Methyl Methacrylate) Scraps and Purification of Depolymerized Products. J. Environ. Manage. 2019, 231, 1012-1020. https://doi.org/10.1016/j.jenvman.2018.10.116.
(18) Kaminsky, W.; Franck, J. Monomer Recovery by Pyrolysis of Poly(Methyl Methacrylate) (PMMA). J. Anal. Appl. Pyrolysis 1991, 19 (C), 311-318. https://doi.org/10.1016/0165-2370(91)80052-A.
(19) A. Gilsdorf, R.; A. Nicki, M.; Y.-X. Chen, E. High Chemical Recyclability of Vinyl Lactone Acrylic Bioplastics. Polym. Chem. 2020, 11 (30), 4942-4950. https://doi.org/10.1039/DOPY00786B.
(20) R. Gowda, R.; Y.-X. Chen, E. Synthesis of β-Methyl-α-Methylene-γ-Butyrolactone from Biorenewable Itaconic Acid. Org. Chem. Front. 2014, 1 (3), 230-234. https://doi.org/10.1039/c3qo00089c.
(21) Tang, X.; Hong, M.; Falivene, L.; Caporaso, L.; Cavallo, L.; Chen, E. Y. X. The Quest for Converting Biorenewable Bifunctional α-Methylene-γ-Butyrolactone into Degradable and Recyclable Polyester: Controlling Vinyl-Addition/Ring-Opening/Cross-Linking Pathways. J. Am. Chem. Soc. 2016, 138 (43), 14326-14337. https://doi.org/10.1021/jacs.6b07974/.
(22) J Macromol, J. H.; Ueda, M.; Takahashi, M.; Imai, Y.; Pittman, C. U. Synthesis and Homopolymerization Kinetics of A-Methylene-δ-Valerolactone, an Exo-Methylene Cyclic Monomer with a Nonplanar Ring System Spanning the Radical Center. J. Polym. Sci., Polym. Lett. Ed 1976, 11 (10), 401.
(23) Xu, T. Q.; Yu, Z. Q.; Zhang, X. M. Recyclable Vinyl-Functionalized Polyesters via Chemoselective Organopolymerization of Bifunctional α-Methylene-δ-Valerolactone. Macromol. Chem. Phys. 2019, 220 (12), 1900150. https://doi.org/10.1002/macp.201900150.
(24) Li, J.; Liu, F.; Liu, Y.; Shen, Y.; Li, Z. Functionalizable and Chemically Recyclable Thermoplastics from Chemoselective Ring-Opening Polymerization of Bio-Renewable Bifunctional α-Methylene-δ-Valerolactone. Angew. Chemie 2022, 134 (32), e202207105. https://doi.org/10.1002/ange.202207105.
(25) Liu, Y.; Kou, X.; Xu, C.; Zhou, W.; Zhang, H.; Liu, F.; Shen, Y.; Li, Z. Chemoselective and Controlled Ring-Opening Copolymerization of Biorenewable α-Methylene-δ-Valerolactone with F-Caprolactone toward Functional Copolyesters. Polym. Chem. 2023, 14 (19), 2326-2332. https://doi.org/10.1039/d3py00188a.
(26) Manzer, L. E. Catalytic Synthesis of α-Methylene-γ-Valerolactone: A Biomass-Derived Acrylic Monomer. Appl. Catal. A Gen. 2004, 272, 249-256. https://doi.org/10.1016/j.apcata.2004.05.048.
(27) Al-Naji, M.; Puértolas, B.; Kumru, B.; Cruz, D.; Bäumel, M.; Schmidt, B. V. K. J.; Tarakina, N. V.; Pérez-Ramírez, J. Sustainable Continuous Flow Valorization of γ-Valerolactone with Trioxane to α-Methylene-γ-Valerolactone over Basic Beta Zeolites. ChemSusChem 2019, 12 (12), 2628-2636. https://doi.org/10.1002/CSSC.201900418.
(28) Hutchenson, K. W.; Kourtakis, K.; Manzer, L. E. U.S. Pat. No. 7,199,254B2—Liquid Phase Synthesis of Methylene Lactones Using Novel Catalyst, 2007.
(29) Hutchenson, K. W.; Kourtakis, K.; Manzer, L. E. U.S. Pat. No. 7,164,032B2—Supercritical Fluid Phase Synthesis of Methylene Lactones Using Novel Catalyst Field of Invention, 2007.
(30) Hattori, H. Heterogeneous Basic Catalysis. Chem. Rev. 1995, 95 (3), 537-558. https://doi.org/10.1021/cr00035A005.
(31) Bond, J. Q.; Martin Alonso, D.; West, R. M.; Dumesic, J. A. γ-Valerolactone Ring-Opening and Decarboxylation over SiO 2/Al2O3 in the Presence of Water. Langmuir 2010, 26 (21), 16291-16298. https://doi.org/10.1021/la101424a.
(32) Wong, C. Y. Y.; Choi, A. W. T.; Lui, M. Y.; Fridrich, B.; Horváth, A. K.; Mika, L. T.; Horváth, I. T. Stability of Gamma-Valerolactone under Neutral, Acidic, and Basic Conditions. Struct. Chem. 2017, 28 (2), 423-429. https://doi.org/10.1007/s11224-016-0887-6.
(33) Marianou, A. A.; Michailof, C. M.; Ipsakis, D. K.; Karakoulia, S. A.; Kalogiannis, K. G.; Yiannoulakis, H.; Triantafyllidis, K. S.; Lappas, A. A. Isomerization of Glucose into Fructose over Natural and Synthetic MgO Catalysts. ACS Sustain. Chem. Eng. 2018, 6 (12), 16459-16470. https://doi.org/10.1021/acssuschemeng.8B03570.

### INCORPORATION BY REFERENCE

All US and PCT patent application publications and US patents mentioned herein are hereby incorporated by reference in their entirety as if each individual patent application publication or patent was specifically and individually indicated to be incorporated by reference. In case of conflict, the present application, including any definitions herein, will control.

### EQUIVALENTS

While specific embodiments of the subject invention have been discussed, the above specification is illustrative and not restrictive. Many variations of the invention will become apparent to those skilled in the art upon review of this specification and the claims below. The full scope of the invention should be determined by reference to the claims, along with their full scope of equivalents, and the specification, along with such variations.

We claim:

1. A method of producing an α,β-unsaturated lactone, comprising:
i. providing a reactor containing a catalyst, wherein the catalyst comprises a metal oxide catalyst; and
ii. contacting the catalyst with a lactone and a carbonyl compound in the reactor, thereby producing the α,β-unsaturated lactone.

2. The method of claim 1, wherein the lactone is δ-valerolactone (DVL).

3. The method of claim 1, wherein the carbonyl compound is formaldehyde or acetaldehyde.

4. The method of claim 1, wherein the α,β-unsaturated lactone is α-methylene-δ-valerolactone (MVL).

5. The method of claim 1, wherein the metal oxide catalyst comprises an alkali metal oxide or alkali earth metal oxide, or a combination of them.

6. The method of claim 5, wherein the metal oxide catalyst comprises lithium, sodium, potassium, beryllium, strontium, magnesium, calcium, barium, or cesium, or a combination of any of them.

7. The method of claim 6, wherein the metal oxide catalyst comprises MgO, BaO, CaO, or a combination of any of them.

8. The method of claim 7, wherein the metal oxide catalyst comprises CaO.

9. The method of claim 1, further comprising an oxide support; wherein the metal oxide catalyst is deposited on the oxide support.

10. The method of claim 9, wherein the metal oxide catalyst loading on the oxide support is 0.5-20 wt %.

11. The method of claim 1, wherein the oxide support is silica, alumina, titanium oxide, zirconium oxide, carbon, or a combination of them.

12. The method of claim 10, wherein the metal oxide catalyst comprises CaO on a SiO₂ support at a loading of about 2-8 wt %.

13. The method of claim 1, wherein the molar ratio of lactone to carbonyl compound is about 1:1 to about 10:1.

14. The method of claim 13, wherein the molar ratio of lactone to carbonyl compound is about 3:1 to about 6:1.

15. The method of claim 1, wherein the reactor is heated to about 200-500° C.

16. The method of claim 15, wherein the reactor is heated to about 300-400° C.

17. The method of claim 1, wherein the reactor is a flow reactor.

18. The method of claim 1, further comprising calcining the catalyst at about 350-500° C., thereby regenerating the catalyst.

19. The method of claim 1, wherein the α,β-unsaturated lactone is produced with conversion of the lactone of less than about 50%.

20. The method of claim 1, wherein the α,β-unsaturated lactone is produced with selectivity of about 80% to about 100% relative to the other reaction products.
